(19) United States
(12) Patent Application Publication
(10) Pub. No.: US 2026/0283548 A1
(43) Pub. Date: Sep. 24, 2026

(54) DATA-DRIVEN SLEEP PLANNING

(71) Applicant: Whoop, Inc., Boston, MA (US)

(72) Inventors: Emily Rachel Capodilupo, Boston, MA (US); Victoria Harrison Lee, Waltham, MA (US)

(21) Appl. No.: 19/573,793

(22) Filed: Mar. 20, 2026

Related U.S. Application Data
(60) Provisional application No. 63/775,111, filed on Mar. 20, 2025.

(51) Int. Cl. A61B 5/00; A61B 5/024; G16H 40/67

(52) U.S. Cl. CPC A61B 5/486; A61B 5/02405; A61B 5/02416; A61B 5/4815; G16H 40/67

(57) ABSTRACT

A sleep coaching system optimizes sleep recommendations for a user based on a composite of available physiological metrics, user goals, and other user context. The system can further enable a user to select a sleep plan that manages trade-offs between various objective outcomes or goals, and to manually adjust a suggested sleep plan to navigate among different goals and outcomes.

### RELATED APPLICATIONS

This application claims priority to U.S. Prov. Pat. App. No. 63/775,111, filed on Mar. 20, 2025, the entire content of which is hereby incorporated by reference.

This application is also related to International Patent App. No. PCT/US26/20150 filed on Mar. 20, 2026, which claims priority to U.S. Prov. Pat. App. No. 63/775,111, filed on Mar. 20, 2025, where the entire content of each of the foregoing is hereby incorporated by reference.

### BACKGROUND

A wearable physiological monitor may be used to track a variety of fitness metrics such as heart rate, respiration rate, and sleep. In this context, various sleep benchmarks such as sleep duration, sleep consistency, sleep type, and the like can support objective, quantitative analysis of sleep. However, algorithmic sleep coaching on this basis frequently fails when numerically optimized sleep targets diverge from a user's personal goals for short-term or long-term outcomes, or when these targets present undesirable or impractical conflicts for users.

There remains a need for improved sleep coaching that balances competing needs for different sleep, fitness, and lifestyle goals, and that permits a user to navigate trade-offs between outcomes when evaluating sleep recommendations.

### SUMMARY

A sleep coaching system optimizes sleep recommendations for a user based on a composite of available physiological metrics, user goals, and other user context. The system can further enable a user to select a sleep plan that manages trade-offs between various objective outcomes or goals, and to manually adjust a suggested sleep plan to navigate among different goals and outcomes.

According to an aspect of the present disclosure, a computer program product comprises computer executable code embodied in a non-transitory computer readable medium that, when executing on one or more computing devices, causes the one or more computing devices to perform steps of receiving physiological data from a wearable device worn substantially continuously by a user, the physiological data including photoplethysmography data, identifying a sleep history based on the physiological data, the sleep history including a plurality of sleep intervals for the user, calculating a sleep need for the user based on a first portion of the physiological data acquired during a first one or more of the plurality of sleep intervals, calculating a sleep consistency for the user based on a second portion of the physiological data acquired during a second one or more of the plurality of sleep intervals, providing an objective metric for evaluating a subsequent sleep interval of the user based on additional physiological data acquired during the subsequent sleep interval, generating a sleep recommendation for the subsequent sleep interval by the user, the sleep recommendation including a bedtime and a waketime selected to optimize the objective metric for the user, and presenting the sleep recommendation to the user in an interactive interface, wherein the interactive interface displays a calculation of the objective metric based on the sleep recommendation, the interactive interface provides a control for the user to make an adjustment to at least one of the bedtime and the waketime, and the interactive interface responds to the adjustment received from the user by recalculating the objective metric based on the adjustment and displaying a recalculated objective metric to the user.

According to some embodiments, the objective metric may be based on two or more sleep metrics. According to some embodiments, the objective metric may be based on a combination of targets for the sleep need and the sleep consistency after the subsequent sleep interval. According to some embodiments, the objective metric may include one or more of a resting heart rate and a heart rate variability for the subsequent sleep interval. According to some embodiments, providing the objective metric may include receiving a selection by the user of the objective metric from among a plurality of available objective metrics. According to some embodiments, the objective metric may include one or more of a recovery metric, a sleep quality metric, and a long-term health metric. According to some embodiments, the sleep recommendation may be adjusted based on a time zone change detected for the user.

According to an aspect of the present disclosure, a method comprises receiving physiological data from a wearable device worn by a user, identifying a sleep history based on the physiological data, the sleep history including a plurality of sleep intervals for the user, calculating a sleep need and a sleep consistency for the user based on the physiological data, receiving a goal for the user, selecting an objective metric for evaluating a subsequent sleep interval of the user based on additional physiological data acquired during the subsequent sleep interval, generating a sleep recommendation for the subsequent sleep interval based on a predicted optimization of the objective metric according to the sleep goal, and presenting the sleep recommendation to the user as a sleep plan in an interactive interface.

According to some embodiments, the objective metric may include one or more of a target sleep consistency, a target sleep duration, and a target sleep composite index based on a combination of sleep consistency and sleep duration. According to some embodiments, the objective metric may include one or more of a target resting heart rate or a target heart rate variability. According to some embodiments, the sleep recommendation may include one or more of a bedtime and a waketime selected to optimize the objective metric for the user. According to some embodiments, the interactive interface may display a prediction of the objective metric based on the sleep recommendation, the interactive interface may provide a control for the user to make an adjustment to at least one of the bedtime and the waketime, and the interactive interface may respond to the adjustment by recalculating the objective metric based on the adjustment and displaying the recalculated objective metric to the user. According to some embodiments, the sleep recommendation may be adjusted based on one or more of an age of the user, a body mass index of the user, a consumption of the user, a strain experienced by the user, and a day of a week. According to some embodiments, the physiological data may include one or more of heart rate data, heart rate variability data, respiratory rate data, blood oxygen saturation data, skin temperature data, accelerometer data, and GPS data. According to some embodiments, the goal may include a long-term goal for the user. According to some embodiments, the goal may include a short-term goal for the next day. According to some embodiments, the goal may include a sleep goal with a target for one or more objective sleep metrics based on sleep performance for a next sleep interval. According to some embodiments, the goal may include a fitness goal with a target for one or more fitness metrics including one or more of a resting heart rate and a heart rate variability. According to some embodiments, the goal may include a target for a self-reported outcome metric including one or more of an energy level, an alertness, and a performance level.

According to an aspect of the present disclosure, a system comprises a physiological monitor configured to continuously acquire physiological data from a user and a server configured to perform steps of identifying a sleep history based on the physiological data, the sleep history including a plurality of sleep intervals for the user, calculating a sleep need and a sleep consistency for the user based on the physiological data, receiving a goal for the user, selecting an objective metric for evaluating a subsequent sleep interval of the user based on additional physiological data acquired during the subsequent sleep interval, generating a sleep recommendation for the subsequent sleep interval based on a predicted optimization of the objective metric according to the sleep goal, and presenting the sleep recommendation to the user as a sleep plan in an interactive interface.

According to an aspect of the present disclosure, a method for calculating a sleep score for a user comprises receiving historical sleep data and physiological data for the user, the historical sleep data including a sleep interval, calculating, from the historical sleep data and the physiological data, an hours-versus-need metric, a sleep consistency metric, a sleep efficiency metric, and a sleep stress metric for the sleep interval, determining, with a short-term model, a short-term score from the hours-versus-need metric, the sleep consistency metric, and the sleep efficiency metric, wherein the short-term model predicts next-day performance, determining, with a long-term model, a long-term score from the hours-versus-need metric, the sleep consistency metric, and the sleep efficiency metric, wherein the long-term model predicts a long-term health outcome, combining the short-term score and the long-term score to obtain a composite score, applying a sleep stress penalty to the composite score based on the sleep stress metric to obtain a penalized score, applying a non-linear transformation to the penalized score to obtain a transformed score, when the hours-versus-need metric is below a threshold, applying an hours-versus-need penalty to the transformed score to obtain the sleep score, and outputting the sleep score.

According to an aspect of the present disclosure, a method for generating a sleep score comprises receiving sleep data associated with the user including an hours-versus-need metric, a sleep consistency metric, and a sleep efficiency metric, determining, with a short-term model, a short-term score from the sleep data, wherein the short-term model predicts next-day performance, determining, with a long-term model, a long-term score from the sleep data, wherein the long-term model predicts a long-term health outcome, combining the long-term score and the short-term score, modifying the score component based on a sleep stress metric to generate a modified score component, transforming the modified score component with a non-linear transformation to generate the sleep score, and outputting the sleep score.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, with emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 illustrates generalized additive mixed models showing associations between sleep performance and sleep consistency.

FIG. 7 illustrates adjusted models with interactions between sleep consistency and sleep performance.

FIG. 8 illustrates a method for obtaining a nightly sleep recommendation.

FIG. 9 illustrates a user interface displaying sleep data.

FIG. 10 shows a method for data-driven sleep planning.

FIG. 11 shows a user interface for a sleep planner as described herein.

FIG. 12 illustrates an algorithm for calculating a sleep score for use with the systems and methods described herein.

### DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose.

Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary.

The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods, such as an hour, a day or more (including acquisition throughout the day and night), etc. More generally, with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics, such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions, such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data, such as optical intensity signals, or processed data therefrom, such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as DRAM, SRAM, EDO RAM), and so forth. Although not depicted, any of the devices or components described herein may include a computer-readable medium or other memory for storing program instructions, data, and the like.

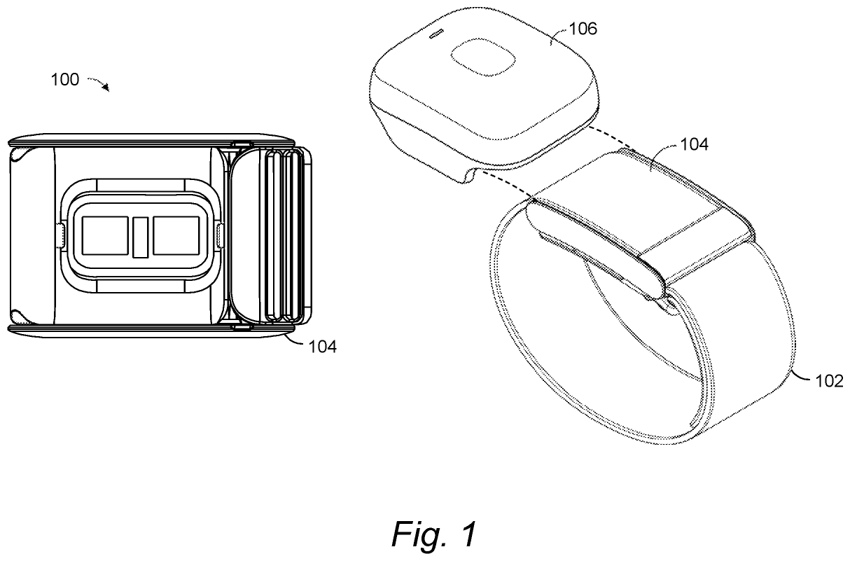

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104, such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light-emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters, including, but not limited to, skin temperature (using a thermometer), galvanic skin response (using a galvanic skin response sensor), motion (using one or more multi-axis accelerometers and/or gyroscope), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communications systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a Global Positioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

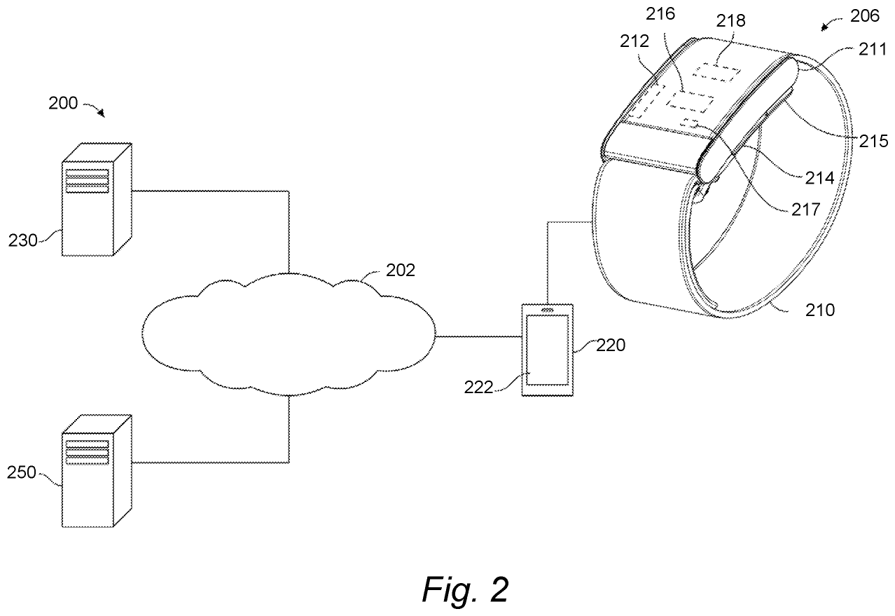

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communication with local resources. By way of non-limiting examples, short-range communications may include Wi-Fi communications, Bluetooth communications, infrared communications, near-field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

In one aspect, the physiological monitor 206 may be configured to continuously or intermittently acquire, store, and/or process physiological data at a frequency sufficient to resolve cardiac activity, including a heart rate and heart rate variability. This may, for example, usefully include sampling rates of 50 Hz (20 ms) or greater on one or more optical channels, as well as synchronized acquisition of temperature data, motion data, and the like. As a significant advantage, this rate of data acquisition can facilitate real-time or near real-time delivery of metrics such as any of the physiological metrics described herein. Similarly, the availability of computational resources, either local or remote, to perform signal processing, embedding, foundation model inferencing, and the like can support real-time coaching or the like to enable timely, actionable user feedback based on the data acquired with the physiological monitor.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

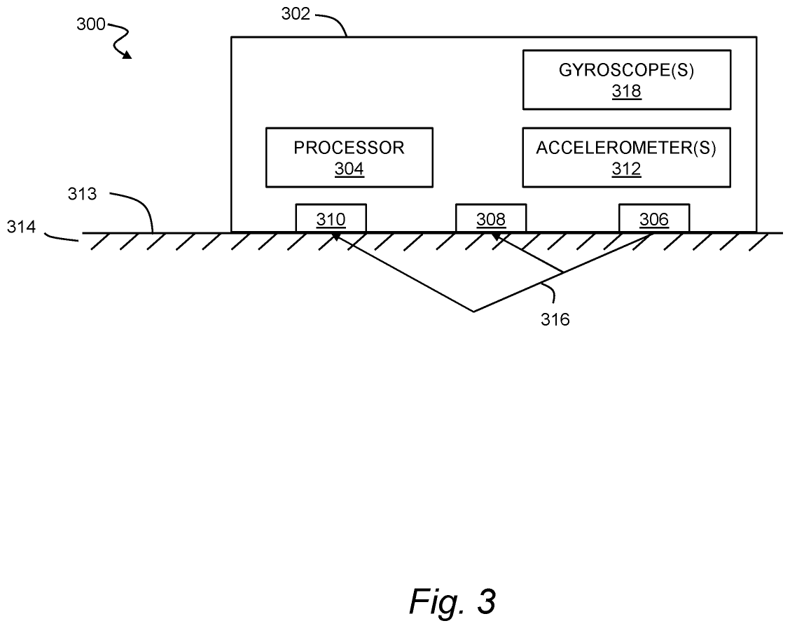

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light emitting diodes that emit light in the green, red, infrared, near infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308, 310 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware-such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

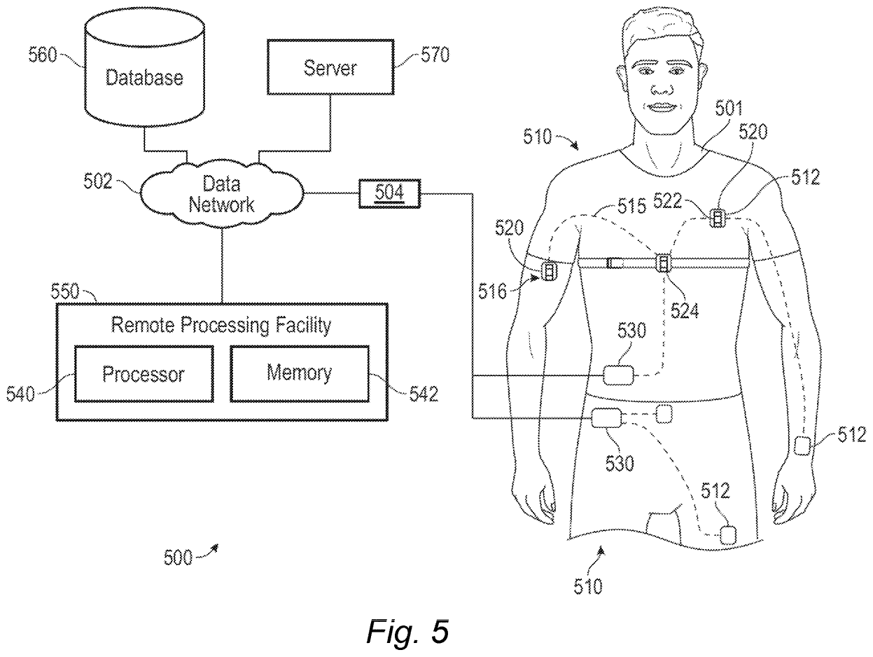

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

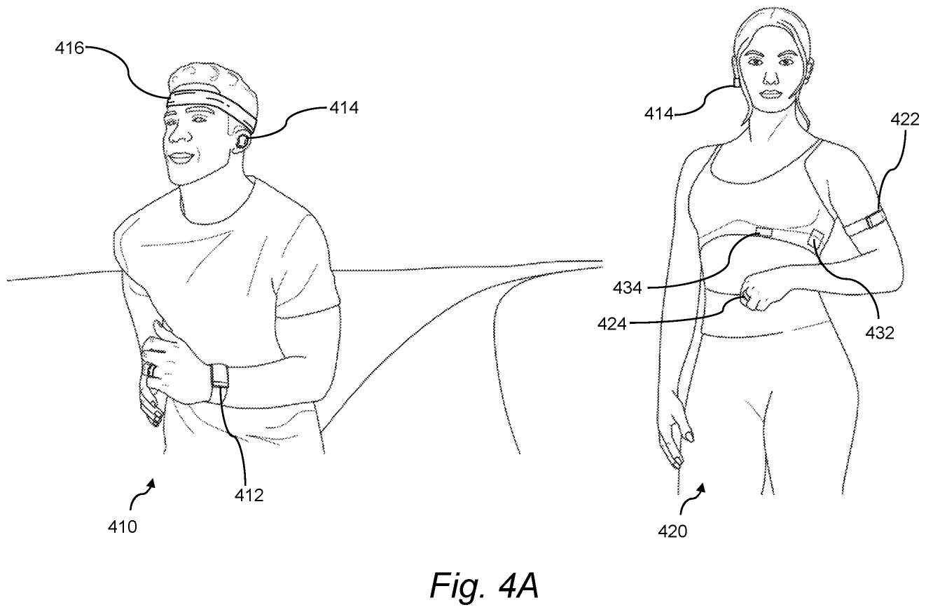

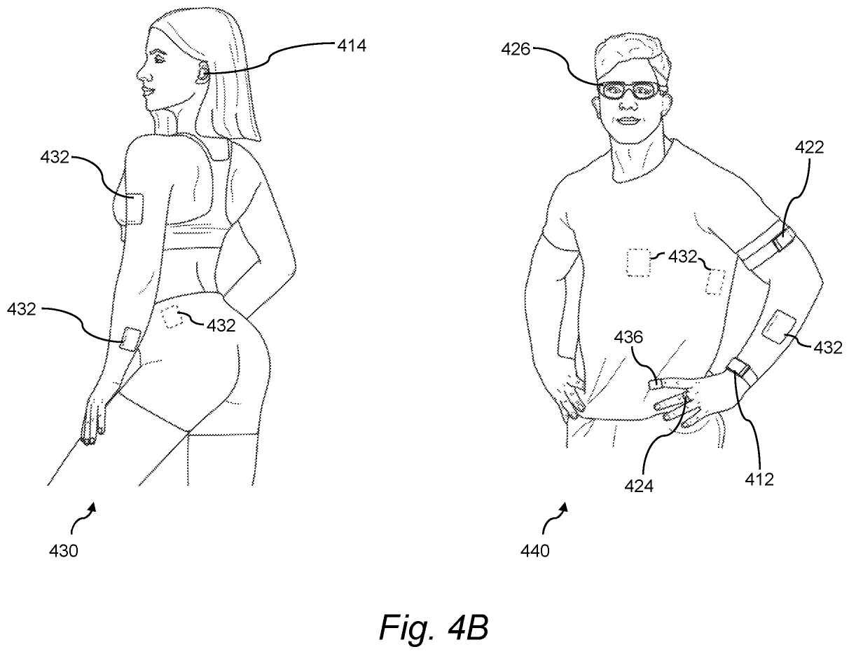

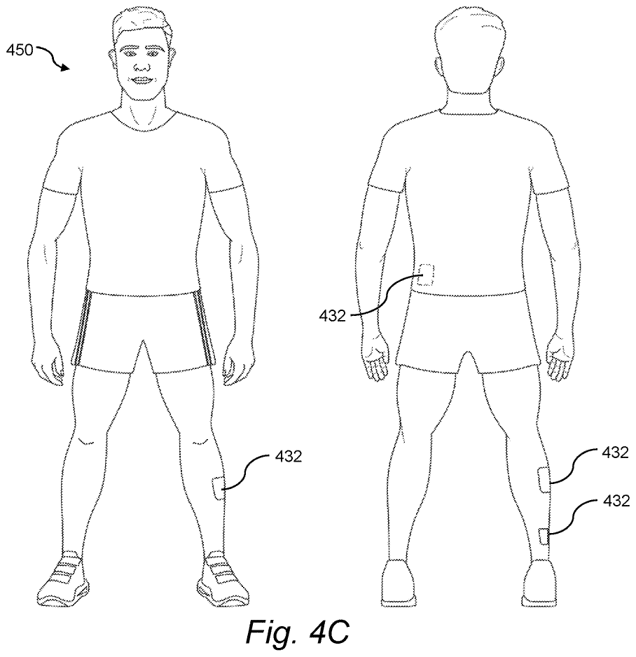

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health, and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes, that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a finger-tip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The finger-tip monitor 436 may include a clamp, clip, or the like, and may be the same or similar to any of the physiological monitors described herein. In some aspects, the finger-tip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications, and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for receiving light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements, such as accelerometer data from the module 520, which may be analyzed by a machine learning model or the like to infer a body position. In another aspect, this may be based on other signals from the module 520. For example, signals from sensors, such as photodiodes, temperature sensors, resistors, capacitors, and the like, may be used alone or in combination to infer a body position. In another aspect, the location may be determined based on the proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data such as any of the data discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources may advantageously be used in combination to improve physiological monitoring and achieve modes of monitoring not previously available.

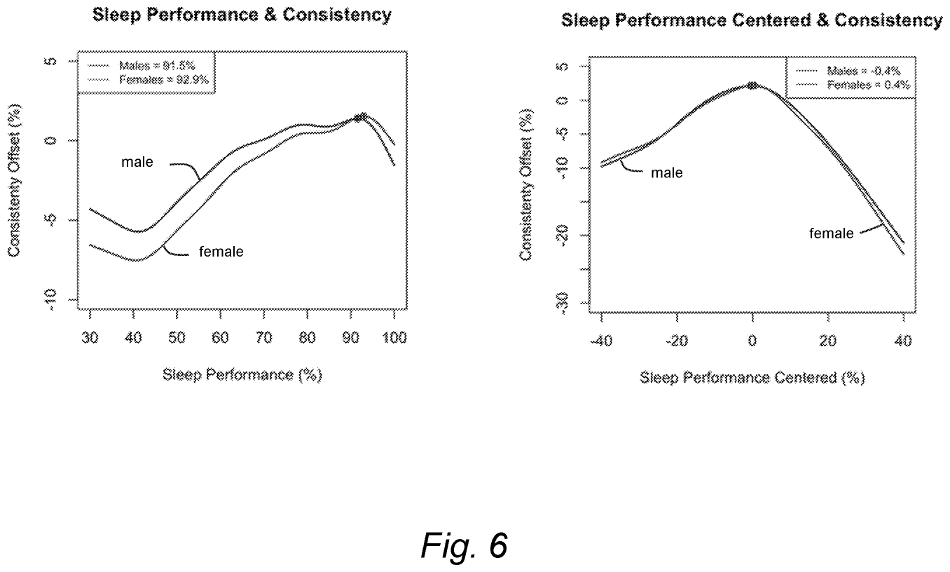

FIG. 6 illustrates generalized additive mixed models showing associations between sleep performance and sleep consistency. More specifically, the figure shows generalized additive mixed models (GAMMs) with associations between sleep performance, where raw values are on the left, and person-mean centered values are on the right. The models are adjusted for age, body mass index, previous day strain and alcohol, month, and weekday or weekend. The dots indicate a location on each curve where sleep consistency is predicted to be optimized for a composite sleep performance score, with the corresponding sleep performance value (raw or centered) provided in the figure legend.

In general, sleep performance and sleep consistency are important contributors to health and performance. However, there is a trade-off between these elements of sleep. In the example illustrated on the left-hand panel of FIG. 6, historical data for a population demonstrates that sleep consistency is currently optimized at 90% sleep performance (actual sleep, as a percentage of sleep need), with additional sleep reducing consistency. The right-hand panel illustrates the within-person effect of altering habitual sleep performance. On the population level, a 10% increase in sleep performance beyond habitual is associated with a 3.5% reduction in consistency, and consistency reductions are amplified with greater increases in performance (e.g., +20% performance=−9% consistency). This quantitatively illustrates a tradeoff between sleep duration and sleep consistency, and the need for a multi-metric model to optimize overnight cardiovascular outcomes for metrics such as resting heart rate (RHR) and heart rate variability (HRV).

It will be understood that sleep consistency and duration may be measured in numerous ways. For example, sleep duration may be measured as a percentage of a current sleep need for the user (which may in turn be an aggregation of a baseline sleep need and any adjustments for accumulated sleep debt and/or naps or other supplemental sleep). Sleep consistency may be measured in a variety of ways. For example, a Sleep Regularity Index (SRI) measures the probability of an individual being in the same state (asleep or awake) at any two time points 24 hours apart, averaged across a period of interest. The SRI is then calculated as:

$$SRI = 100 \times ( 1 - \frac{2 D}{N} )$$

where D is the number of state mismatches and N is the total number of time points compared. In general, an SRI ranges from 0 for completely unaligned sleep patterns to 100 for perfectly regular sleep patterns. Sleep consistency may also or instead be measured using a sleep consistency score that assesses the likelihood that a user's state (sleep or wake) is the same at a particular clock time over a longer interval, such as four consecutive days, which provides a useful and more stable objective measure of sleep regularity. More generally, the SRI may be calculated over a multi-day interval by using a weighted average or other mean or aggregation of multiple days of sleep data to smooth changes in the SRI over time. Other objective metrics may also or instead be used, such as sleep onset regularity (based on consistency of bedtimes), sleep offset regularity (based on predictability of wake-up times), sleep midpoint regularity (based on consistency of the midpoint between sleep onset and waking), and sleep duration regularity (based on the stability of sleep duration).

Although specific embodiments using duration and consistency (and a composite of these two) are described, other objective sleep metrics may also or instead be used as targets for optimizing a sleep plan, provided they can be meaningfully correlated to a measurable target physiological metric, such as heart rate, respiration rate, heart rate variability, body temperature, and so forth. For example, a quality of sleep may be calculated based on a number and duration of sleep interruptions, regularity of breathing, amount of motion, amount and distribution of slow wave sleep, amount and distribution of REM sleep, and so forth, usefully correlated to sleep outcomes, and may be used as the basis for an objective sleep metric that can be used as a coaching and optimization benchmark as contemplated herein.

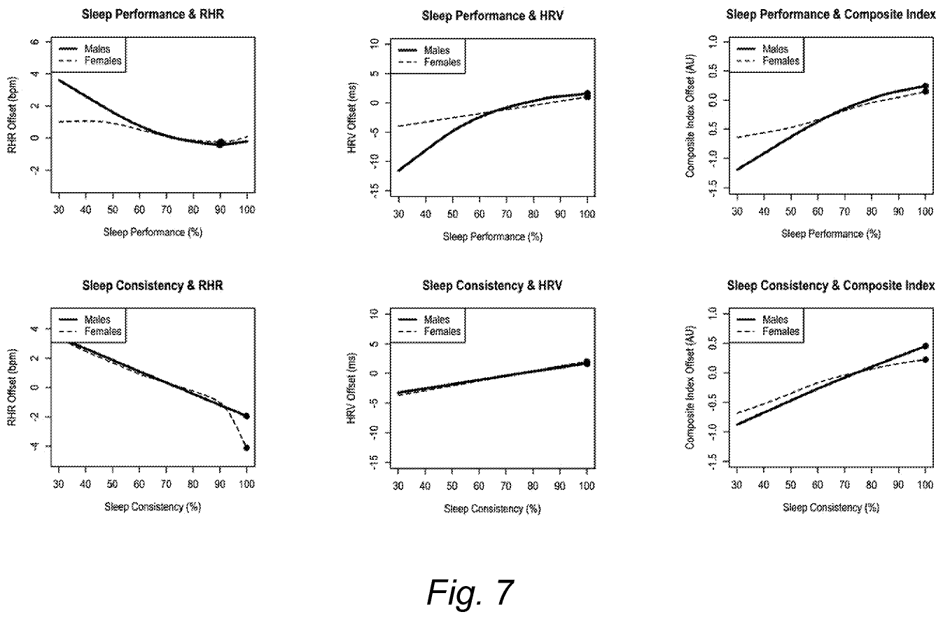

FIG. 7 illustrates adjusted models with interactions between sleep consistency and sleep performance. More specifically, FIG. 7 illustrates generalized additive mixed models showing associations between sleep performance (top) and sleep consistency (bottom) with cardiovascular outcomes in males and females. All models included main effects and the interaction of sleep performance and consistency, and were adjusted for age, BMI, previous day strain and alcohol, month, and weekday or weekend. The dots indicate the location on each curve where cardiovascular metrics are predicted to be optimized, with the corresponding sleep parameter value provided in the figure legend. More specifically, the series of GAMMs in FIG. 7 illustrate sleep performance (top panel) and sleep consistency (bottom panel) predicting RHR and HRV. Models for males and females are shown separately, and all models are adjusted for relevant biological metrics (e.g., age and body mass index [BMI]), lifestyle factors (previous day alcohol [only days where members logged were included] and strain), and temporal variables (month and weekday or weekend). The models generally include the interaction between sleep performance and consistency to account for the association between these variables depicted in FIG. 6.

The GAMMs in FIG. 7 support numerous insights useful for providing sleep coaching and recommendations. In one aspect, RHR was optimized at ~90% sleep performance & 100% consistency, independent of sex. The HRV was optimized at 100% sleep performance & 100% consistency, independent of sex. A composite index that combines RHR and HRV was optimized at 100% sleep performance & 100% consistency, independent of sex. In general, sex differences in associations between sleep performance and cardiovascular outcomes, particularly HRV, were observed. For example, poor sleep performance (<60%) had a more detrimental impact on cardiovascular metrics in males, compared with females, and increasing high-end sleep consistency (90→100%) yielded a substantial and beneficial reduction in RHR in females, by about 2 bpm.

In one aspect, a significant interaction of sleep performance and sleep consistency was detected in RHR models, and both performance and consistency remained independently predictive. These interactions suggest various general guidelines for sleep that may be used as a starting point for analyzing sleep and providing coaching recommendations to optimize outcomes based on RHR. For example, in order to manage a recovery based on RHR, a target or recommendation for a peak result (RHR decrease of 1.0 to 1.5 bpm) may be sleep performance>80% and sleep consistency>80%. A target for an elevated result (RHR decrease of 0.5 to 1.0 bpm) may be a sleep performance of 70-80% and a sleep consistency of 70-80%. A target recommendation for an adequate result (no change in RHR) may be a sleep performance of 60-70% and a sleep consistency of 60-70%.

Similarly, for HRV models, a significant interaction of sleep performance and sleep consistency was detected, and both performance and consistency remained independently predictive. However, compared to RHR models, performance, rather than consistency, appeared to be a more influential predictor of HRV, as is indicated by the more vertical lines in the plot. A notable exception to this trend was observed in females with poor sleep performance (~40-60%), where sleep consistency seemed to be highly influential in predicting HRV outcomes. The HRV models thus suggest various guidelines for sleep that may be used as a starting point for analyzing sleep and providing coaching recommendations to optimize outcomes based on HRV. For example, in order to manage a recovery based on HRV, a target or recommendation for a peak result (HRV increase of 2.0 to 3.0 ms) may be sleep performance>95% and sleep consistency>70% (although the impact of consistency appears small in this case). A target for an elevated result (HRV increase of 0.5 to 2.0 ms) may be a sleep performance of 70-95% and a consistency>70%. A target recommendation for an adequate result (no change in HRV) may be a sleep performance of 60-70% and a sleep consistency of 60-70%.

For a composite index based on HRV and RHR, a significant interaction of sleep performance and sleep consistency was detected, and both performance and consistency remained independently predictive. Though not identical, the diagonal lines on the plot are similar to what was observed for RHR Offset, highlighting the similar importance of sleep performance and sleep consistency in predicting the composite index. The composite models thus suggest coaching recommendations to optimize outcomes based on the composite index. For example, in order to manage a recovery based on the composite index, a target or recommendation for a peak result (composite index increase of 0.4 to 0.6) may be sleep performance>85% and sleep consistency>85%. A target for an elevated result (composite index increase of 0.2 to 0.4) may be a sleep performance of 70-85% and a sleep consistency>70-85%. A target recommendation for an adequate result (no change in HRV) may be a sleep performance of 60-70% and a sleep consistency of 60-70%.

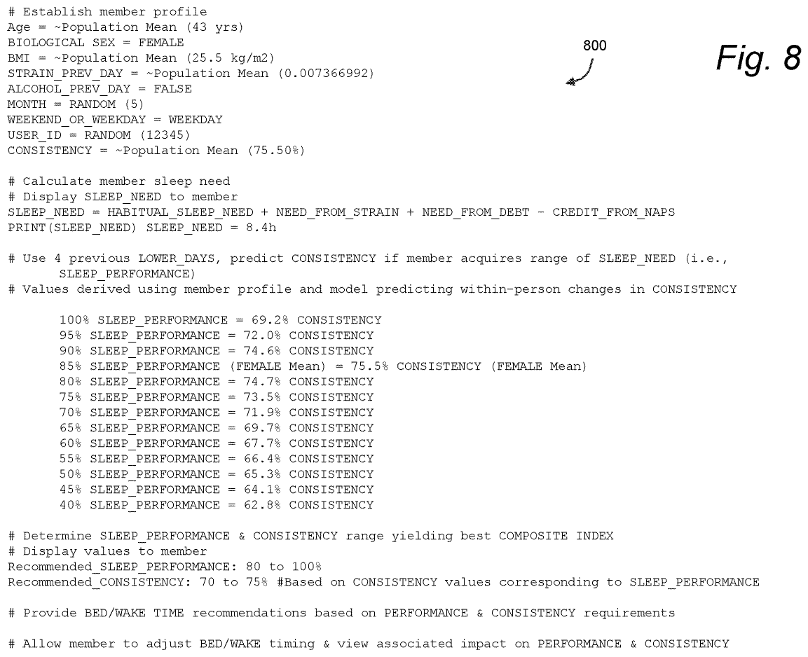

FIG. 8 illustrates a method for obtaining a nightly sleep recommendation. In this case, pseudocode is used to express the manner in which the foregoing relationships may be used to obtain a nightly sleep recommendation. The method 800 starts with a basic equation for calculating sleep need, and calculates a sleep consistency that would be anticipated to result from that need based on a target outcome. The process then determines whether reducing sleep need might increase consistency to an extent that would be predicted to improve an objective overnight recovery variable (i.e., RHR and HRV). The optimal sleep need and consistency combination, and associated bed/wake times, are then presented to the user. The user may then be provided with an opportunity to adjust a bedtime and/or waketime and view the anticipated impact of these changes on the recovery variable(s). In general, these instructions may be used to generate corresponding program code, or may be provided to a large language model or other inferencing engine, along with user data for sleep history, to generate a corresponding sleep plan. This latter approach also facilitates an interactive user session for viewing and revising a sleep plan according to a user's other lifestyle constraints, fitness goals, and so forth.

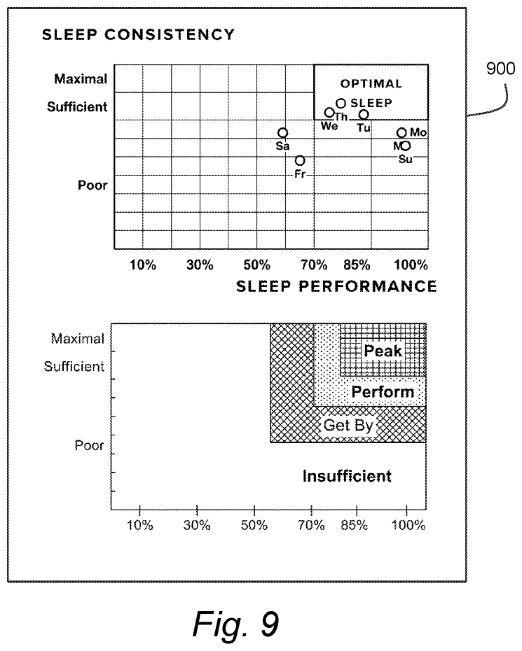

FIG. 9 illustrates a user interface displaying sleep data. In one aspect, the user interface 900 may provide a chart of sleep history for the user with sleep consistency on one axis and sleep performance (e.g., percentage of sleep need) on another axis. This may helpfully convey to a user the dual importance of consistency and duration of sleep by explicitly displaying zones of optimal sleep. One or more optimal sleep zones may be outlined on the chart to provide a visual depiction of how the recent sleep history for the user (e.g., a plot of daily sleep results for a preceding seven days or a preceding week) compares to general sleep objectives. A general outline of sleep objectives may also or instead be displayed, illustrated as composite zones of duration and consistency for categories of sleep quality such as peak performance, performance, getting by, or insufficient. A specific recommendation may also be provided, and may be placed in a separate graph (e.g., the lower panel) as one or more points showing specific sleep objectives for the user. This may include supplemental information for sleep planning such as time to bed, time to wake, total time in bed, total sleep time, and so forth. The user interface 900 may be personalized for a user in numerous ways, such as by accounting for typical sleep interruptions for the user, time to fall asleep for the user, a current sleep deficit for the user, and so forth.

Specific sleep recommendations may also be adapted in a number of ways to a particular user or context. For example, where user-specific information on typical sleep patterns is available (e.g., the amount of restorative sleep, time in bed relative to hours of sleep, number of interruptions), this personal information may be used to adjust sleep recommendations to achieve sleep objectives for that user. Recommendations may also be adapted, e.g., to provide gradual transitions to accommodate travel, daylight savings changes, sunrise and sunset data, and so forth, in order to increase consistency in the presence of external changes.

In another aspect, the user interface may support interactive sleep planning by providing user inputs in order to modify or explore sleep recommendations. In one aspect, the user may be provided with options to optimize on different sleep metrics such as specific next-day outcomes (e.g., a recovery score, or a target for RHR or HRV), sleep objectives (100% of sleep need, maximum consistency, catch up on sleep deficit, specific bed time, specific wake-up time, short-term recovery, long-term health, and so forth), which may be optimized based on the user history and general relationships among sleep and other metrics to provide an optimized recommendation. This approach may advantageously leverage years of continuous monitoring data for a particular user including heart rate data, respiration data, sleep patterns, strain and recovery scores, and user logging of subjective feelings (tired, rested, focused, . . . ) and external events (late caffeine, evening exercise, late meal, alcohol consumption, illness, . . . ). In another aspect, the user may be provided with controls to vary sleep parameters such as time in bed and time awake, and the user interface may display the impact of these changes on user outcomes. This permits the user to modify a plan in objectively sub-optimal but subjectively optimal ways for the user. For example, where the user is willing to sacrifice some sleep consistency in order to catch up on lost sleep, or where the user wishes to wake up at a specific time regardless of total sleep and/or impact on sleep-based outcomes, the user may be permitted to enter these revisions to a sleep plan and review estimates of resulting, qualitative outcomes.

More generally, any techniques for managing tradeoffs between various sleep metrics, both in terms of providing an initial recommendation or range of recommendations to a user, and/or permitting a user to vary the recommended sleep schedule in order to explore impacts of variations, may be used to provide a multi-variable, user-adjustable sleep coaching platform as contemplated herein.

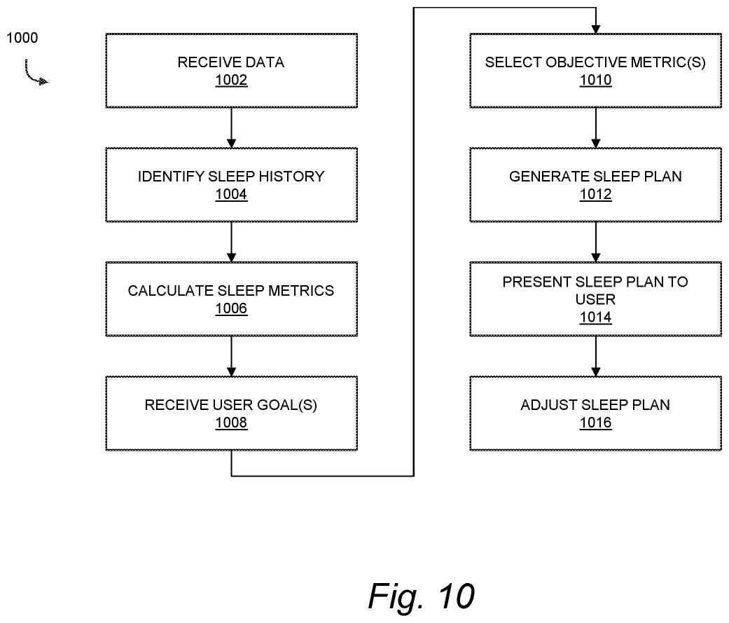

FIG. 10 shows a method for data-driven sleep planning. The method 1000 may be used with any of the other systems and methods described herein, and may in general support data-driven sleep planning for a user by creating a sleep plan for the user to achieve measurable, objective user goals, while permitting optimization among multiple competing sleep outcomes and supporting user customization with an interactive interface that facilitates user investigation of different sleep scenarios.

As shown in step 1002, the method 1000 may include receiving physiological data from a wearable device worn by a user. In some embodiments, the wearable device may acquire physiological data continuously or periodically while the user engages in daily activity and sleep. The physiological data may include, for example, heart rate data, heart rate variability data, respiratory rate data, blood oxygen saturation data, skin temperature data, accelerometer data, GPS data, or any combination thereof. The physiological data may be received from a physiological monitor, a user device, a remote server, or any combination thereof, and may be stored for subsequent analysis of sleep behavior and prediction of outcomes for a subsequent sleep interval.

As shown in step 1004, the method 1000 may include identifying a sleep history based on the physiological data. The sleep history may include a plurality of sleep intervals for the user. In some embodiments, identifying the sleep history may include detecting sleep onset, wake time, total sleep time, time in bed, interruptions, or other characteristics of individual sleep intervals over a plurality of days. The sleep history may be used to characterize historical sleep timing, duration, and regularity for the user, and may further provide user-specific context for generating a sleep recommendation for a subsequent sleep interval.

As shown in step 1006, the method 1000 may include calculating sleep metrics. For example, this may include calculating a sleep need and a sleep consistency for the user based on the physiological data. In some embodiments, the sleep need may correspond to an estimated amount of sleep appropriate for the user for a forthcoming sleep interval, and may account for baseline sleep need, sleep deficit, naps, or other supplemental sleep. The sleep consistency may correspond to a degree of regularity in the timing or pattern of the user's sleep across the sleep history. By way of example, sleep consistency may be based on a sleep regularity index, a multi-day sleep consistency score, sleep onset regularity, sleep offset regularity, sleep midpoint regularity, sleep duration regularity, or another metric that characterizes regularity of sleep behavior over time.

As shown in step 1008, the method 1000 may include receiving a goal for the user. In some embodiments, the goal may include a long-term goal for the user, a short-term goal for the next day, a sleep goal with a target for one or more objective sleep metrics based on sleep performance for a next sleep interval, a fitness goal with a target for one or more fitness metrics, or a target for a self-reported outcome metric. For example, the goal may include a target associated with sleep consistency, sleep duration, a sleep composite index, resting heart rate, heart rate variability, energy level, alertness, performance level, or any combination thereof.

As shown in step 1010, the method 1000 may include selecting an objective metric for evaluating a subsequent sleep interval of the user based on additional physiological data acquired during the subsequent sleep interval. In some embodiments, the objective metric may be selected according to the received goal and may be used as a basis for optimizing a sleep plan for the subsequent sleep interval. In some cases, the objective metric may be the goal itself. For example, a user may wish to optimize sleep performance, sleep duration, or some other sleep metric directly, or another fitness metric such as resting heart rate. Other goals may be more general goals, such as peak performance, alertness, energy, or total awake time. In these cases, the objective metric may be one or more physiological metrics that contribute to the desired goal, e.g., in the form of a high recovery or sleep score calculated based on observable, objective physiological data. The objective metric may include one or more target sleep metrics, such as a target sleep consistency, a target sleep duration, or a target sleep composite index based on a combination of sleep consistency and sleep duration. The objective metric may also or instead include one or more physiological or fitness-related metrics, such as a target resting heart rate or a target heart rate variability.

In some embodiments, the method 1000 may include selecting among a plurality of different optimization targets before or during presentation of the sleep plan. For example, the user may choose to optimize an objective metric such as a next-day recovery variable, a target for resting heart rate, a target for heart rate variability, a sleep objective of achieving a target percentage of sleep need, a goal of increasing consistency, a goal of reducing sleep deficit, or another objective associated with the user goal. The method 1000 may then modify the selected objective metric and corresponding sleep recommendation based on the selected optimization target.

As shown in step 1012, the method 1000 may include generating a sleep plan. In general, the sleep plan may be a sleep recommendation for the subsequent sleep interval based on a predicted optimization of the objective metric according to the sleep goal. In some embodiments, generating the sleep recommendation may include evaluating expected trade-offs between sleep duration and sleep consistency in view of the selected objective metric. The sleep recommendation may include one or more recommended sleep plan parameters, such as a bedtime and a waketime for a sleep plan selected to optimize the objective metric for the user. The sleep recommendation may also be adjusted based on one or more contextual or user-specific factors, including age of the user, body mass index of the user, consumption of the user, strain experienced by the user, a day of a week, typical sleep interruptions, time to fall asleep, current sleep deficit, travel, daylight savings changes, sunrise data, sunset data, or any combination thereof.

As shown in step 1014, the method 1000 may include presenting the sleep recommendation to the user as a sleep plan in an interactive interface. In some embodiments, the interactive interface may display a prediction of the objective metric based on the sleep recommendation, along with supplemental sleep planning information, such as a recommended bedtime, a recommended waketime, total time in bed, total sleep time, or any combination thereof. The interactive interface may present the sleep recommendation as one or more specific sleep objectives for the user and may permit the user to review trade-offs among multiple outcomes or goals when evaluating the recommended sleep plan.

As shown in step 1016, the method 1000 may include adjusting the sleep plan, e.g., in an interactive user session or other automated or manual adjustment process. In some embodiments, the method 1000 may further include receiving a user adjustment to at least one parameter of the sleep plan through a control in the interactive interface. For example, the interactive interface may provide a control that permits the user to adjust at least one of the bedtime and the waketime. The adjustment may reflect a user preference, a lifestyle constraint, a desired wake time, a desire to prioritize additional sleep duration, a desire to prioritize sleep consistency, or another user-specific consideration. In another aspect, the adjustments may be received in a chat interface or other foundation model configured to provide sleep coaching to the user. The foundation model may usefully be configured to use data for the user in order to adapt recommendations to the user's actual sleep history and other user context.

In some embodiments, responsive to the user adjustment, the method 1000 may further include recalculating the objective metric based on the adjustment and displaying the recalculated objective metric to the user in the interactive interface. In this manner, the user may explore how changes to time in bed, wake time, or another sleep parameter are predicted to affect the selected objective metric or related outcomes. The recalculated objective metric may be presented as an updated prediction for a sleep metric, a physiological metric, a fitness-related metric, or any combination thereof.

In some embodiments, the method 1000 may iteratively repeat one or more of steps 1010, 1012, and 1014 after the user adjustment in order to update the objective metric, regenerate the sleep recommendation, and present a revised sleep plan. This iterative process may permit the user to navigate trade-offs among short-term recovery, long-term health, sleep regularity, sleep duration, subjective outcomes, and scheduling constraints while still receiving objective feedback based on the user's physiological data and sleep history.

In some embodiments, the method 1000 may continue until the user accepts a current sleep plan, a threshold condition for optimization is satisfied, or a sleep interval begins. The accepted sleep plan may then be used as a basis for evaluating the subsequent sleep interval using the additional physiological data acquired during that subsequent sleep interval. In this manner, the method 1000 may provide a multi-variable, user-adjustable sleep coaching process that balances sleep duration, sleep consistency, physiological outcomes, fitness outcomes, and user preferences.

According to the foregoing, in one aspect, there is disclosed herein a system comprising a physiological monitor and a server. The physiological monitor may be configured to continuously acquire physiological data from a user. The server may be configured to perform steps of: identifying a sleep history based on the physiological data, the sleep history including a plurality of sleep intervals for the user; calculating a sleep need and a sleep consistency for the user based on the physiological data; receiving a goal for the user; selecting an objective metric for evaluating a subsequent sleep interval of the user based on additional physiological data acquired during the subsequent sleep interval; generating a sleep recommendation for the subsequent sleep interval based on a predicted optimization of the objective metric according to the sleep goal; and presenting the sleep recommendation to the user as a sleep plan in an interactive interface.

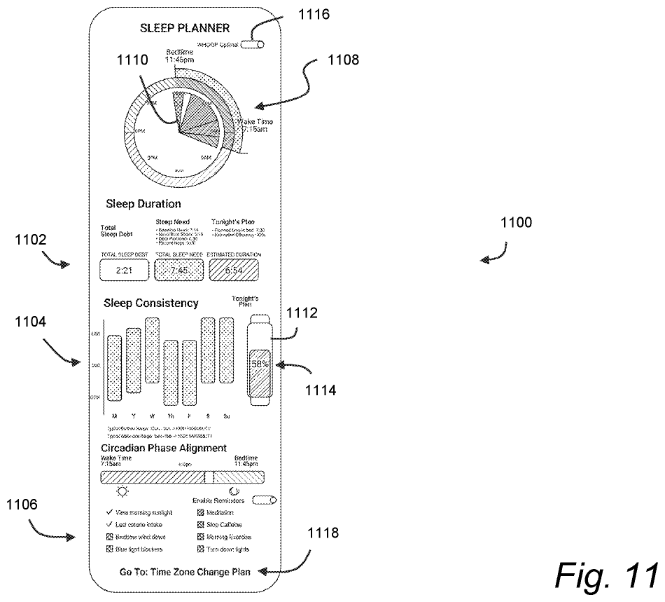

FIG. 11 shows a user interface for a sleep planner as described herein. In general, the user interface 1100 may support bedtime and waketime planning for a user based on the principles described herein.

In one aspect, the user interface 1100 may present sleep history and sleep pattern information to assist a user in assessing current sleep needs. For example, the user interface 1100 may display sleep metrics 1102 such as a current sleep debt, a current sleep need, and a current sleep duration expected for the current sleep plan. The user interface 1100 may also display a sleep history 1104 including, e.g., a week's worth of prior sleep data as a bar chart showing bedtimes and waketimes. The user interface 1100 may also display recommendations 1106 such as behavioral recommendations for a current night. This may, for example, include bedtime wind-down intervals, meditation, caffeine avoidance, alcohol avoidance, and so forth. These recommendations may be based on known or inferred user goals or sleep needs or any other factor(s).

The user interface 1100 may include one or more user controls 1108 that can be operated by a user to enter a sleep plan or modify an existing sleep plan. This may, for example, include one or more dials, sliders, clock arms, or the like for entry and modification to a bedtime and a waketime. This collection of user controls 1108 may also include an informational display of statistical sleep data 1110 such as an angular histogram or other distribution of bedtimes and waketimes over some historical interval. This can assist a user in determining whether and how a current sleep plan compares to historical sleep patterns for the user. The user interface 1100 may also display a sleep plan 1112 currently configured for the user, e.g., as a bar or other representation of a bedtime and waketime, along with one or more objective metrics 1114 predicted based on the current sleep plan. This may, for example, include a sleep efficiency, sleep performance, sleep duration, sleep score, or other objective sleep metric, and/or any of the physiological metrics or other objectively measurable goals that a user might select for use in sleep planning.

The user interface 1100 may also include a variety of other tools and controls to support interactive sleep planning. For example, the user interface 1100 may include an optimization control 1116 configured to override current customer or automatic sleep plan results and calculate a sleep plan optimized for one or more metrics of interest. This optimization control 1116 may permit a user to select a particular metric or combination of metrics for optimization, and/or may default to a particular metric or set of metrics with a known association with short-term or long-term goals of interest. The user interface 1100 may also or instead include a control 1118 for accessing a time zone change planner to assist the user in easing an anticipated change to a different time zone. More generally, any data, controls, inputs, or outputs useful for assisting a user in sleep planning may be incorporated into an interactive sleep planning user interface as described herein.

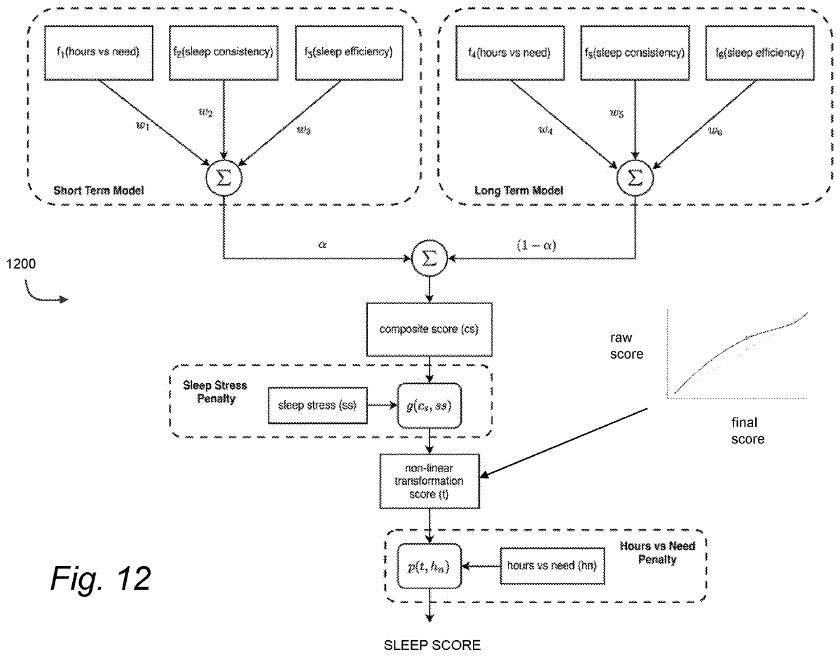

FIG. 12 illustrates an algorithm for calculating a sleep score for use with the systems and methods described herein. While any of a number of objective sleep metrics may be used with the systems and methods described herein, the following technique usefully integrates a number of sleep variables into a single objective metric that provides a useful, integrated view of sleep performance that accounts for short-term and long-term sleep patterns. As a significant advantage, the techniques described herein may be used with a metric such as the sleep score to automatically generate a sleep plan with a highly favorable and/or quantitatively optimized predicted outcome for a user. The process 1200 may be executed by one or more processors of a sleep coaching system using historical sleep data, physiological data, and one or more user profile variables to identify a sleep plan predicted to improve one or more recovery-related outcomes.

In some embodiments, the process 1200 may begin by establishing a member profile for the user. The member profile may include one or more biological, lifestyle, and temporal variables used by one or more predictive models. By way of example, the member profile may include age, biological sex, body mass index, previous-day strain, previous-day alcohol consumption, month, weekday or weekend status, a user identifier, or any combination thereof. These inputs may be used to evaluate individualized relationships between sleep variables and one or more outcome variables, thus facilitating model selection or adaptation to individual users or user types. The calculation scoring process then involves four primary stages: evaluating base models, calculating a composite score, applying conditional penalties, and performing non-linear transformations.

In embodiments, the base model may include a short-term model implemented as a Generalized Additive Model (GAM), to determine the relationship of hours versus need (f1), sleep consistency (f2), and sleep efficiency (f3) to next-day performance. This model controls for physiological and demographic variables, including strain, gender, age, and BMI. The components are multiplied by respective weights (w1, w2, and w3) and aggregated via a summation process to produce a short-term score. For example, next-day performance can be represented by a composite index based on person-mean centered z-scores of inverted resting heart rate and heart rate variability, such that higher values correspond to better-than-usual physiology. The short-term model output can be computed as a weighted combination of partial dependence values for the component metrics, with hours versus sleep need weighted at 0.55, sleep consistency weighted at 0.10, and sleep efficiency weighted at 0.35. This portion of the model can account for short-term changes in sleep outcomes resulting from nightly sleep behaviors.

In parallel, a long-term model, also implemented as a GAM, calculates the relationship of hours versus need (f4), sleep consistency (f5), and sleep efficiency (f6) to long-term health outcomes. Similar to the short-term model, it controls for strain, gender, age, and BMI. These variables are multiplied by heuristically derived weights (w4, w5, and w6) and aggregated via a summation process to generate a long-term score. In some embodiments, the long-term health outcome can be represented by average resting heart rate over a longer interval, such as a year-long interval. The long-term model output can be computed as a weighted combination of partial dependence values for the component metrics, with hours versus sleep need weighted at 0.3, sleep consistency weighted at 0.4, and sleep efficiency weighted at 0.3. This portion of the model can account for longer-term changes in sleep outcomes resulting from nightly sleep behaviors.

In some embodiments, the partial dependence values used in the short-term model and the long-term model may be post-processed to include saturation thresholds and slope extension below minimum modeled values. For example, the partial dependence values can saturate at 100% for hours versus sleep need, 90% for sleep consistency, and 90% for sleep efficiency, such that increases beyond those thresholds do not further increase the corresponding model contribution. In some embodiments, a positive slope may be extended below a lower modeled range for one or more component metrics so that improvements from low values still produce an increase in the sleep performance score.

A composite score (cs) may then be generated by combining the outputs of the short-term and long-term models using a weighted sum. An alpha parameter determines the relative contribution of each model. Specifically, the short-term model is weighted by alpha, while the long-term model is weighted by 1−alpha. In one embodiment, alpha is set to 0.3 to place a greater emphasis on long-term health outcomes.

Following the generation of the composite score (cs), a sleep stress penalty may be applied based on a sleep stress (ss) input. This is executed via a function, g(cs, ss), which applies a percentage-based deduction based on the percentage of time spent in high sleep stress. This penalty is conditionally applied only if the high sleep stress is at or above 1%, and it is capped at a maximum deduction of 60%.

The penalized score may then undergo a non-linear mapping process to output a non-linear transformation score (t). For example, the non-linear transformation can be configured such that, when the four sleep component metrics fall within the same category, the resulting sleep performance score also falls within that category. In some embodiments, the category boundaries can include a high sleep performance category at 85% or greater, an adequate sleep performance category from 70% to 84%, and a low sleep performance category below 70%. In some embodiments, sleep consistency can be categorized as high at 80% or greater, adequate from 70% to 79%, and low below 70%; sleep efficiency can be categorized as high at 90% or greater, adequate from 80% to 89%, and low below 80%; and sleep stress can be categorized as high at or below 1%, adequate from 1% to 5%, and low above 5%. In general, this mapping helps to ensure that when all four analyzed subcomponents (hours versus need, consistency, efficiency, and sleep stress) fall into a unified categorization band—such as Optimal, Sufficient, or Poor—the resulting performance score will also fall within that same category.

Finally, an hours versus need penalty may be applied to the transformed score (t) using the original hours versus need input (hn) via a function p(t, hn). This applies a percentage-based penalty if the hours versus need metric is below 70%, subject to a maximum deduction of 60%. This step ensures that extremely short sleeps are treated as detrimental to health outcomes and appropriately penalizes consistently short sleep durations, regardless of their consistency or efficiency.

Overall, the process 1200 may be configured so that a user can achieve an upper sleep performance score when the component metrics meet or exceed the associated saturation thresholds and the user has less than 1% high sleep stress. For example, a user can achieve an upper score when hours versus sleep need is at least 100%, sleep consistency is at least 90%, sleep efficiency is at least 90%, and high sleep stress is below 1%. In some embodiments, the process 1200 can operate in a sleep consistency calibration mode for a user lacking adequate consistency data. In such embodiments, an alternative model can be used in which sleep consistency is omitted from the short-term and long-term model outputs. For example, the short-term model may use weights of 0.61 for hours versus sleep need, 0 for sleep consistency, and 0.39 for sleep efficiency, and the long-term model may use weights of 0.5 for hours versus sleep need, 0 for sleep consistency, and 0.5 for sleep efficiency. In some embodiments, a different transformation curve may also be used during the calibration mode.

The resulting sleep score usefully combines short-term and long-term sleep history, while adjusting for relevant sleep factors such as sleep stress and sleep performance. This provides a useful analytical anchor for estimating the result of an upcoming sleep interval in order to identify an optimal sleep plan for a user, and/or assist a user in tailoring a sleep plan to user-provided constraints such as a fixed bedtime, fixed waketime, hours available for sleep, performance expectations for the following day, and so forth. The process 1200 may thus provide a sleep score for presentation to the user, storage, transmission to one or more downstream services, or use in one or more sleep coaching, recovery, or health-monitoring processes.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software.

Thus, in one aspect, each method described above, and combinations thereof, may be embodied in computer-executable code that, when executing on one or more computing devices, performs the steps thereof. In another aspect, the methods may be embodied in systems that perform the steps thereof, and may be distributed across devices in a number of ways, or all of the functionalities may be integrated into a dedicated, standalone device or other hardware. The code may be stored in a non-transitory fashion in a computer memory, which may be a memory from which the program executes (such as random access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine, to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A computer program product comprising computer executable code embodied in a non-transitory computer readable medium that, when executing on one or more computing devices, causes the one or more computing devices to perform steps of:
receiving physiological data from a wearable device worn substantially continuously by a user, the physiological data including photoplethysmography data;
identifying a sleep history based on the physiological data, the sleep history including a plurality of sleep intervals for the user;
calculating a sleep need for the user based on a first portion of the physiological data acquired during a first one or more of the plurality of sleep intervals;
calculating a sleep consistency for the user based on a second portion of the physiological data acquired during a second one or more of the plurality of sleep intervals;
providing an objective metric for evaluating a subsequent sleep interval of the user based on additional physiological data acquired during the subsequent sleep interval;
generating a sleep recommendation for the subsequent sleep interval by the user, the sleep recommendation including a bedtime and a waketime selected to optimize the objective metric for the user; and
presenting the sleep recommendation to the user in an interactive interface, wherein:
the interactive interface displays a calculation of the objective metric based on the sleep recommendation,
the interactive interface provides a control for the user to make an adjustment to at least one of the bedtime and the waketime, and
the interactive interface responds to the adjustment received from the user by recalculating the objective metric based on the adjustment and displaying a recalculated objective metric to the user.

2. The computer program product of claim 1, wherein the objective metric is based on two or more sleep metrics.

3. The computer program product of claim 1, wherein the objective metric is based on a combination of targets for the sleep need and the sleep consistency after the subsequent sleep interval.

4. The computer program product of claim 1, wherein the objective metric includes one or more of a resting heart rate and a heart rate variability for the subsequent sleep interval.

5. The computer program product of claim 1, wherein providing the objective metric includes receiving a selection by the user of the objective metric from among a plurality of available objective metrics.

6. The computer program product of claim 1, wherein the objective metric includes one or more of a recovery metric, a sleep quality metric, and a long-term health metric.

7. The computer program product of claim 1, wherein the sleep recommendation is adjusted based on a time zone change detected for the user.

8. A method comprising:
receiving physiological data from a wearable device worn by a user;
identifying a sleep history based on the physiological data, the sleep history including a plurality of sleep intervals for the user;
calculating a sleep need and a sleep consistency for the user based on the physiological data;
receiving a goal for the user;
selecting an objective metric for evaluating a subsequent sleep interval of the user based on additional physiological data acquired during the subsequent sleep interval;
generating a sleep recommendation for the subsequent sleep interval based on a predicted optimization of the objective metric according to the goal; and
presenting the sleep recommendation to the user as a sleep plan in an interactive interface.

9. The method of claim 8, wherein the objective metric includes one or more of a target sleep consistency, a target sleep duration, and a target sleep composite index based on a combination of sleep consistency and sleep duration.

10. The method of claim 8, wherein the objective metric includes one or more of a target resting heart rate or a target heart rate variability.

11. The method of claim 8, wherein the sleep recommendation includes one or more of a bedtime and a waketime selected to optimize the objective metric for the user.

12. The method of claim 11, wherein:
the interactive interface displays a prediction of the objective metric based on the sleep recommendation,
the interactive interface provides a control for the user to make an adjustment to at least one of the bedtime and the waketime, and
the interactive interface responds to the adjustment by recalculating the objective metric based on the adjustment and displaying a recalculated objective metric to the user.

13. The method of claim 8, wherein the sleep recommendation is adjusted based on one or more of an age of the user, a body mass index of the user, a consumption of the user, a strain experienced by the user, and a day of a week.

14. The method of claim 8, wherein the physiological data includes one or more of heart rate data, heart rate variability data, respiratory rate data, blood oxygen saturation data, skin temperature data, accelerometer data, and GPS data.

15. The method of claim 8, wherein the goal includes a long-term goal for the user.

16. The method of claim 8, wherein the goal includes a short-term goal for the next day.

17. The method of claim 8, wherein the goal includes a sleep goal with a target for one or more objective sleep metrics based on sleep performance for a next sleep interval.

18. The method of claim 8, wherein the goal includes a fitness goal with a target for one or more fitness metrics including one or more of a resting heart rate and a heart rate variability.

19. The method of claim 8, wherein the goal includes a target for a self-reported outcome metric including one or more of an energy level, an alertness, and a performance level.

20. A system comprising:
a physiological monitor configured to continuously acquire physiological data from a user; and
a server configured to perform steps of:
identifying a sleep history based on the physiological data, the sleep history including a plurality of sleep intervals for the user;
calculating a sleep need and a sleep consistency for the user based on the physiological data;
receiving a goal for the user;
selecting an objective metric for evaluating a subsequent sleep interval of the user based on additional physiological data acquired during the subsequent sleep interval;
generating a sleep recommendation for the subsequent sleep interval based on a predicted optimization of the objective metric according to the goal; and
presenting the sleep recommendation to the user as a sleep plan in an interactive interface.
